(19) United States
(12) Patent Application Publication
(10) Pub. No.: US 2024/0266005 A1
(43) Pub. Date: Aug. 8, 2024

(54) MACHINE LEARNING PLATFORM FOR FINDING SOLID CATALYSTS FOR DEPOLYMERIZATION REACTIONS

(71) Applicant: X Development LLC, Mountain View, CA (US)

(72) Inventors: Alexander Holiday, Brookfiled, WI (US); Vahe Gharakhanyan, Mountain View, CA (US); Falak Shah, Mountain View, CA (US); Nisarg Vyas, Mountain View, CA (US); Tusharkumar Gadhiya, Gandhinagar (IN)

(73) Assignee: X Development LLC, Mountain View, CA (US)

(21) Appl. No.: 18/435,957

(22) Filed: Feb. 7, 2024

Related U.S. Application Data
(60) Provisional application No. 63/483,807, filed on Feb. 8, 2023.

(51) Int. Cl. G16C 20/10; G16C 20/30; G16C 20/70; G16C 20/80

(52) U.S. Cl. CPC G16C 20/10; G16C 20/30; G16C 20/70; G16C 20/80

(57) ABSTRACT

A computational platform for generating solid catalysts for depolymerization reactions is described. The platform may include a first generative model to determine synthesizable crystal structures that could be used as solid catalysts for depolymerization. The first generative model may determine synthesizability and/or stability of solid catalysts. The first generative model may take in voxel representations of a crystal structure and then use a variational autoencoder to encode into latent space. The first generative model may also include a property learning component to determine synthesizable crystals in latent space. Candidate materials may then be identified in the latent space and then decoded into a blurred voxel representation. The blurred voxel representation may be transformed to a crystal structure. The platform may include a second generational model for identifying crystal surfaces and/or adsorption sites. Adsorption energies can be predicted and solid catalyst candidates for depolymerization can be identified.

### CROSS-REFERENCE TO RELATED APPLICATIONS

This application claims priority to U.S. Provisional Patent Application No. 63/483,807, filed on Feb. 8, 2023, titled “MACHINE LEARNING PLATFORM FOR FINDING SOLID CATALYSTS FOR DEPOLYMERIZATION REACTIONS,” which is incorporated by reference in its entirety for all purposes.

### BACKGROUND

Chemical recycling aims to break down plastic waste into the monomeric building blocks it was produced from, enabling a circular economy in which polymers are produced from chemically recycled plastics instead of relying on nonrenewable inputs derived from petroleum. Plastic recycling may include the conversion of waste plastics (polyethylene terephthalate (PET), polylactic acid (PLA)) into their monomer components (bis(2-hydroxyethyl) terephthalate (BHET), lactate, respectively) to replace virgin plastics derived from oil. Ionic liquids (ILs) are a highly tunable class of chemicals that has shown a promising ability to depolymerize plastics in liquid-phase depolymerization reactions, but it's unclear how to navigate the large solid catalyst design space to improve reaction yields and find prospective solid state catalysts for specific depolymerization reactions.

Selecting a specific solid catalyst to use for depolymerization is a challenging task. Whether a particular solid catalyst is synthesizable may not be known. Additionally, if a solid catalyst is known to be synthesizable, the suitability of the solid catalyst for depolymerization may also not be known. Experimentally verifying possible solid catalysts is time consuming and resource intensive. Therefore, it would be advantageous to have a computational and/or modeling technique that may identify particular solid catalysts that are predicted to be synthesizable and to have desirable properties for depolymerization. Such techniques may identify new compounds with significantly fewer resources and on more realistic timescales. The candidate solid catalysts may be used to more efficiently and/or effectively depolymerize polymers.

### BRIEF SUMMARY

Embodiments include a machine learning-based computational platform for finding solid catalysts for depolymerization reactions. The proposed platform may include a first generative model to determine synthesizable crystal structures that could be used as solid catalysts for depolymerization. The first generative model may determine synthesizability using formation energy and energy above the convex hull. The first generative model may take in voxel representations of a crystal structure and then use a variational autoencoder to encode into latent space. The first generative model may also include a property learning component to determine synthesizable crystals in latent space. Candidate materials may then be identified in the latent space and then decoded into a blurred voxel representation. The blurred voxel representation may be encoded and decoded to generate a crystal structure. A second generative model may be used to determine a crystal surface and to generate adsorption sites of the crystal structure. The results of the first and second generative models may be inputted into an adsorption energy prediction framework. The adsorption energy prediction framework can identify solid catalyst candidates for depolymerization. The framework may determine possible adsorption sites based on a desired adsorption energy range.

Methods may include accessing a latent space that relates voxel representations of crystal structures to values for one or more metrics. Each of the one or more metrics may characterize synthesizability or stability of the crystal structures. Methods may in addition include identifying a region or position in the latent space having a predicted metric below or above a cutoff value. Methods may also include transforming the identified region in the latent space into at least one candidate crystal structure. Methods may further include outputting a representation of the at least one candidate crystal structure as a synthesizable crystal. Other embodiments of this aspect include corresponding computer systems, apparatus, and computer programs recorded on one or more computer storage devices, each configured to perform the actions of the methods.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 shows a platform for generating solid catalyst candidates for depolymerization according to embodiments of the present invention.

FIG. 2 shows a model architecture for generating new crystalline structures according to embodiments of the present invention.

FIG. 3 illustrates an example of how individual points in the embedding space can correspond to a given voxel representation and to predicted properties according to embodiments of the present invention.

FIG. 4 shows an ordered latent space in the learned property of formation energy according to embodiments of the present invention.

FIG. 5 is a flowchart of an example process 500 for predicting synthesizable crystals according to embodiments of the present invention.

FIG. 6 is an illustrative architecture of a computing system implemented as some embodiments of the present invention.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

Embodiments described herein include a machine learning-based computational platform for identifying and/or generating solid catalysts for depolymerization reactions. Effectively and efficiently depolymerizing polymers is important for plastic recycling. The parameter space of possible catalysts to aid in depolymerization is huge and likely includes some yet-to-be-discovered catalysts. Solid catalysts, which can be represented in the simplest form as crystals, might be used for depolymerization. Rapidly synthesizing and analyzing solid catalysts experimentally is challenging, if not impossible. Machine learning techniques can be used to screen possible solid catalysts and identify specific solid catalysts for further study and/or experimental analysis. Such machine learning techniques may not have been previously widely applied to depolymerization because depolymerization problem was thought to be too complicated, even if the parameter space of possible solid catalysts was reduced. However, a focus on adsorption energy rather than kinetic rate parameters and mechanisms may allow for the machine learning techniques to be useful in identifying candidate solid catalysts for depolymerization. The combination of machine learning techniques described herein has also not been previously used for identifying solid catalysts for depolymerization.

The terms “cutoff” and “threshold” refer to predetermined numbers used in an operation. For example, a cutoff size can refer to a size above which fragments are excluded. A threshold value may be a value above or below which a particular classification applies. Either of these terms can be used in either of these contexts. A cutoff or threshold may be “a reference value” or derived from a reference value that is representative of a particular classification or discriminates between two or more classifications. Such a reference value can be determined in various ways, as will be appreciated by the skilled person. For example, metrics can be determined for two different cohorts of materials with different known classifications, and a reference value can be selected as representative of one classification (e.g., a mean) or a value that is between two clusters of the metrics (e.g., chosen to obtain a desired sensitivity and specificity). As another example, a reference value can be determined based on statistical analyses or simulations of samples.

The term “machine learning model” may include a model that learns to transform input data to a prediction. A machine learning model may have been trained via supervised learning, which may use labeled training data (e.g., where the machine learning model learns to predict the labels). Alternatively, a machine learning model may have been trained via unsupervised learning, which may use unlabeled data (e.g., and the model may learn to detect one or more structures, patterns, etc. in the unlabeled data). Machine learning models often are developed, trained, and/or implemented using a computer or a processor. Machine learning models may include statistical models.

The term “generative model” may refer to a model that learns to generate/output new inputs (e.g., new images, new text, new materials). Generative models are in contrast to “regression” models, which take the inputs and map them to some prediction, like the presence of a cat in an image or the adsorption energy of a molecule on a catalyst. The “prediction” in a generative model may be a new surface or a new crystal system. The “prediction” in a regression model may be adsorption energy.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

### I. Solid Catalyst Generation Platform

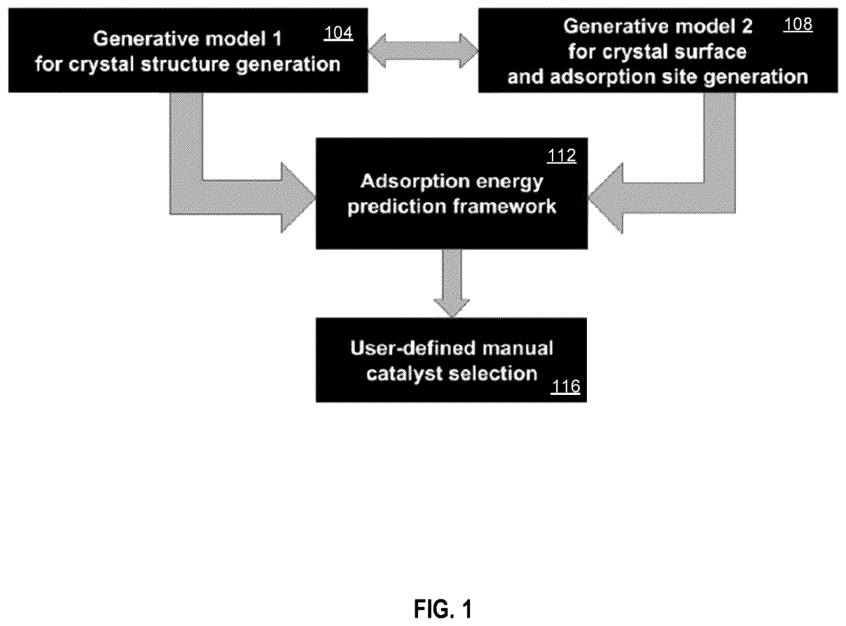

FIG. 1 shows a platform for generating solid catalyst candidates for depolymerization. The platform uses a first generative model 104 for generating crystal structures for candidate solid catalysts and a second generative model 108 for predicting crystal surfaces and/or adsorption sites of the candidate solid catalysts. At adsorption energy prediction framework 112, adsorption energy is predicted using the generated crystal surface and/or adsorption site. User-defined manual catalyst selection 116 may involve filtering candidate catalysts based on adsorption energy. A summary of the platform follows. Additional details of components are described in sections I.A-I.E below.

First generative model 104 generates crystal structures by predicting crystal structures that are synthesizable. The synthesizability criterion is chosen to be a negative formation energy and some threshold value (e.g., 26 meV) for the energy above convex hull. We can also construct our model by assuming that the materials in our database that have been synthesized are synthesizable, and those that have not been attempted are not (note that the latter assumption may not hold, as the fact that a particular crystal simply has not yet been synthesized does not mean that it cannot be synthesized). First generative model 104 is described in detail in section I.A. Briefly, first generative model 104 may be trained with existing crystal structure data and synthesizability data. First generative model 104 may relate crystal structure data and synthesizability data in a latent space. Regions or positions in the latent space with high predicted synthesizability may be identified and transformed into crystal structures. First generative model 104 may differ from other crystal structure generative models at least by including a property learning component, where the property learned relates to synthesizability (formation energy, energy above convex hull or synthesizability tag). The predicted properties are then analyzed after the model is run to determine if the crystal structure is synthesizable and/or stable. Embodiments described herein may involve predicting synthesizability and/or stability with the same model used to generate new crystal structures.

Second generative model 108 generates representations of crystal surfaces and adsorption sites. Representations of the crystal surfaces may include identities of the atoms in the crystal surface, bond lengths, or bond angles. Second generative model 108 may generate a fingerprint that includes some part of FTCP (Fourier-Transformed Crystal Properties) representation (know which crystal is generated), hkl indices or one-hot encoded indices (know surface proposed for adsorption), and/or xyz coordinates of adsorption site(s) (know exact point of contact) Second generative model 108 is described in more detail in section I.B. In some embodiments, second generative model 108 may use as an input the generated crystal structures from first generative model 104. For example, the crystal surface may be generated from analyzing a candidate crystal structure and determining what surfaces are likely to be present based on the characteristics (e.g., bond energies, bond lengths, atoms) of the crystal structure.

In some embodiments, second generative model 108 may generate representations of crystal surfaces and/or adsorption sites without the crystal structure generated from first generative model 104. Second generative model 108 may be trained with existing crystal surface data and adsorption energy. Second generative model 108 may relate crystal surface data and the adsorption energy in a latent space. Regions or positions in the latent space with desired adsorption energy may be identified and transformed into crystal surfaces. In some embodiments, second generative model 108 may relate crystal surface data and the adsorption energy data without using a latent space.

Second generative model 108 may also predict characteristics of the adsorption sites on the crystal surfaces. An adsorption site is a location on the crystal surface where a polymer or a polymer subunit (e.g., monomer, dimer, trimer, or oligomer) may attach (or locations if there are multiple points of contact). While the polymer or polymer is attached to the adsorption site, a reactant may react with the polymer or polymer subunit to decompose the polymer or polymer subunit. Ideally, the adsorption of the polymer to the crystal surface increases the probability or ease of reaction. Characteristics of the adsorption sites may include the band structure, identities of atoms on the surface, bond angles, dangling bonds, or unpaired electrons at or near the adsorption sites.

Using first generative model 104 and second generative model 108 together may allow for information to be transferred to the other model and aid in further generation. For example, first generative model 104 may determine crystals with specific symmetry and/or atoms are synthesizable. Second generative model 108 can then be updated to include these specific symmetries and/or atoms, or results from the second generative model 108 can be filtered to include crystal surfaces with the specific crystal symmetries and/or atoms. The combination of first generative model 104 and second generative model 108 may result in a more efficient generation of candidate crystal structures than using only a single generative model.

First generative model 104 may use a larger reference dataset to cover a larger materials space compared to second generative model 108. Second generative model 108 may include a smaller dataset that may be applied to only specific adsorbate system and possibly a limited materials space.

Adsorption energy prediction framework 112 estimates adsorption energies for a polymer, monomer, dimer, or oligomer related to a crystal surface and/or adsorption site. Details of the adsorption energy prediction framework are described in section I.C. Adsorption energy prediction framework 112 may be created using publicly available small molecule adsorption on catalyst surface databases. These databases include The Open Catalyst Project (Jain et al., APL materials (2013); Chanussot et al., ACS Catalysis (2021)). These databases may be updated so that small adsorbates in the databases are replaced with monomers, dimers, or trimers. In some embodiments, a new database for adsorption energy and polymers may be created using Density Functional Theory (DFT), as described in The Open Catalyst Project.

Adsorption energy prediction framework 112 may use the outputs of both first generative model 104 and second generative model 108. For example, adsorption energy prediction framework 112 may estimate adsorption energy when first generative model 104 indicates a synthesizable crystal structure and second generative model 108 indicates a crystal surface and an adsorption site related to the crystal structure.

In some embodiments, adsorption energy prediction framework 112 may not use both first generative model 104 and second generative model 108. For example, adsorption energy prediction framework 112 may predict the most appropriate adsorption energy related to a generated crystal structure from first generative model 104 without the generated crystal surface or adsorption site from second generative model 108. Additionally, and more likely, adsorption energy prediction framework 112 may predict an adsorption energy related to a generated crystal surface and adsorption site from second generative model 108 without the generated crystal structure from first generative model 104.

In some embodiments, second generative model 108 may include adsorption energy prediction framework 112. Second generative model 108 may directly generate surfaces and/or sites with desired adsorption energies.

User-defined manual catalyst selection 116 may involve filtering candidate catalysts based on adsorption energy. Details of catalyst selection are described in section I.D. Adsorption energy prediction framework 112 or second generative model 108 may generate a library of predicted adsorption energies for several catalysts. The most promising candidates for depolymerization may be selected from the library based on adsorption energy and/or other criteria. For example, the adsorption energy may be within a certain target range. The target range may be an adsorption energy that is not too strong so as to overly limit desorption of the product and not too weak so as to overly limit activation of the reactant (i.e., at a Sabatier optimum energy). In embodiments, the catalyst(s) may be selected by a computing device based on user-defined criteria.

The manually selected catalysts from user-defined manual catalyst selection 116 can be tested both computationally and experimentally, if possible, for synthesizability, adsorption energy, and suitability for depolymerization.

### A. Crystal Structure Generative Model

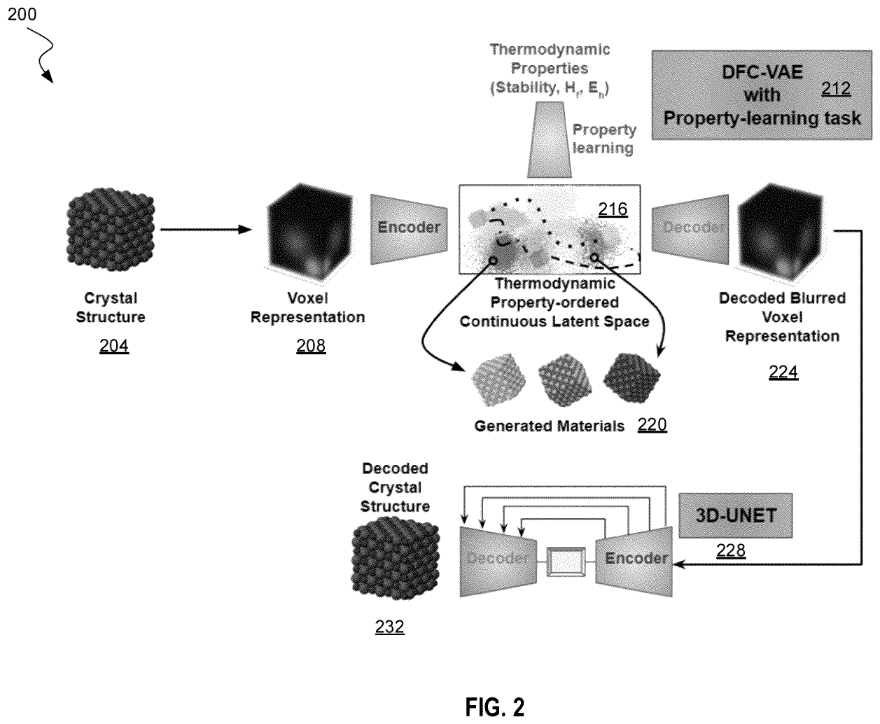

FIG. 2 shows a model architecture 200 for generating new crystalline structures. Model architecture 200 may be used in first generative model 104. Model architecture 200 involves generating voxel representations and embedding the voxel representations with synthesizability data in a latent space. The latent space includes learning to determine regions with high synthesizability. Blurred voxel representations from the regions are generated. The blurred voxel representations are then decoded into a crystal structure.

At stage 204, crystal structure data is obtained. A materials database may be the source of the crystal structure data. For example, the database may be the Material Project (Jain, APL Materials (2013)). Crystallographic Information Files (CIFs) may be obtained for different crystal structures. CIFs are described in Hall et al., Acta Cryst. (1991), the entire contents of which are incorporated herein for all purposes.

At stage 208, a voxel representation of the crystal structure is generated using the CIFs. The voxel representation may be a voxel mass density field. As an example, a 5 Angstrom cubic cell with a 32×32×32 voxel grid may represent a crystal structure. The voxel representation may include different matrices for each crystal structure. Matrices may include a matrix with the local electron density of atoms, a matrix with the identity of each atom, a matrix with the atomic occupancy status of each voxel, a matrix with unit cell length, and/or a matrix with coordinates of each voxel. The voxel representations may be generated using techniques described by Court et al., J. of Chemical Information and Modeling (2020) or Hoffman et al., arXiv: 1909.00949 (2019), the entire contents of both of which are incorporated herein by reference for all purposes.

At stage 212, a variational autoencoder with the property-learning embeds crystal mass density fields into a latent space 216. A deep feature-consistent variational autoencoder with the property-learning task (DFC-prop-VAE) is used to learn the identity map of voxel-represented mass density fields and the associated atom coordinates. The encoder and decoder architectures include a property learning task to determine a binary synthesizability measure and/or other properties, which may include formation energy and energy above convex hull. As an example, the property learning task may include two fully-connected layers for binary synthesizability measure, energy above the convex hull, and formation energy prediction. Formation energy may refer to the energy required for or released while generating a specific crystal structure when referenced to some other energy of constituents. Energy above the convex hull may refer to the formation energy difference between the crystal and a convex hull trace of related crystals. Energy above the convex hull may be called hull energy. A crystal may be considered synthesizable if experimental data on the crystal exists. For example, a crystal may be considered synthesizable if the crystal is present in the Inorganic Crystal Structure Database (ICSD), which is maintained by the National Institute of Standards and Technology (NIST).

Instead of DFC-prop-VAE, generative models to learn the identity map of a crystal representation and relate the latent space to synthesizability properties to generate synthesizable crystals may include models from Noh et al., Matter (2019) and Ren et al., Matter (2022). Particularly, the crystal representations and model architectures can take the forms described in either Noh et al., Matter (2019) and Ren et al., Matter (2022) with a property-learning task attached to the latent space to predict the properties of interest to us (i.e., formation energy, energy above hull and/or synthesizability tag).

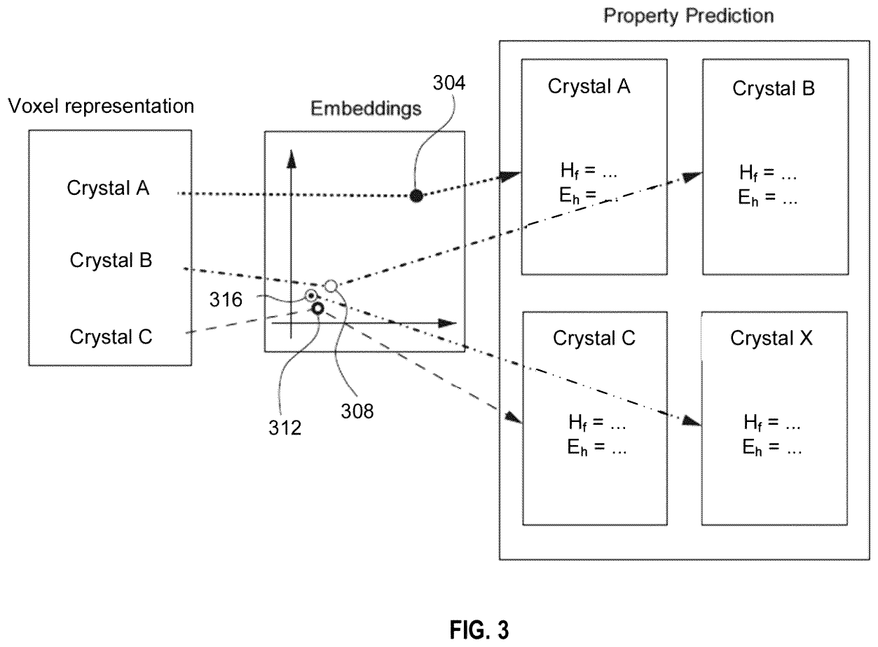

FIG. 3 illustrates an example of how individual points in the embedding space can correspond to a given voxel representation (that may be predicted using a reconstruction Decoder) and to predicted properties. In embodiments, the generative model may refer to a model that generates an output, identifying catalysts (e.g., crystal structures) that are not yet known to be useful for a particular context (e.g., depolymerization). The predicted properties may be identified using another model (e.g., a regression model on top of a generative model, that translates a given position in the embedding space into a predicted property).

In some instances, the first generative model can be configured to generate multiple outputs for any given position within an embedding space, where the multiple outputs correspond to multiple properties. For example, the model may generate a predicted formation energy and hull energy for a given position in the embedding space. In some instances, multiple models are used, where each model generates predictions corresponding to a single property. For example, a first model may predict a formation energy, and a second model may predict a hull energy.

In the embedding-space representation shown in FIG. 3, data points 304, 308, and 312 are shown. These data points are associated with measured values of properties, and data point 316 is associated with no measured property value. In the depicted example, Crystal A (represented by data point 304) is predicted to have unfavorable properties for synthesizability, while Crystal C (represented by data point 312) and Crystal B (represented by data point 308) are predicted to have favorable properties for synthesizability. After the property learning task was used to predict properties in the embedding space, a representation of promising Crystal X (represented by data point 316) is identified. Data point 316 is close in the embedding space to data points 308 and 312. Given that crystals that are represented by data points 308 and 312 have favorable properties, the proximity of data point 316 suggests that the crystal represented by data point 316 will also have favorable properties. The “promising crystal” can correspond to a position in the embedding space where a condition associated with each predicted property is satisfied.

For example, a promising crystal can be associated with a predicted formation energy below or above a formation energy threshold and a hull energy below hull energy threshold. That is, predictions generated by the generative model(s) can be used to identify positions within the embedding space that correspond to desired properties of interest (e.g., predicted formation energies that are below/above a predefined formation energy threshold, hull energies that are below a predefined hull energy threshold).

As another example, a score may be generated for each of some or all of the crystals represented by a position within the embedding space. The score may be defined to be a weighted average of z-scores that is based on predicted properties of interest. The values of the predicted properties of interest may be normalized. A particular score (e.g., higher or lower than a cutoff) may indicate a more promising crystal for synthesizability and/or stability. To illustrate, the score may be defined to be a weighted average of the predicted normalized formation energy or the negative of a predicted normalized formation energy, the negative or positive of a predicted normalized hull energy (including log hull energy). One or more promising catalysts may be identified as those corresponding to the n lowest scores (e.g., as corresponding to the lowest score or as corresponding to any of the lowest 10 scores) or as those corresponding to a score below an absolute threshold. Alternatively, one or more promising catalysts may be identified as those corresponding to the n highest scores (e.g., as corresponding to the highest score or as corresponding to any of the highest 10 scores) or as those corresponding to a score above an absolute threshold.

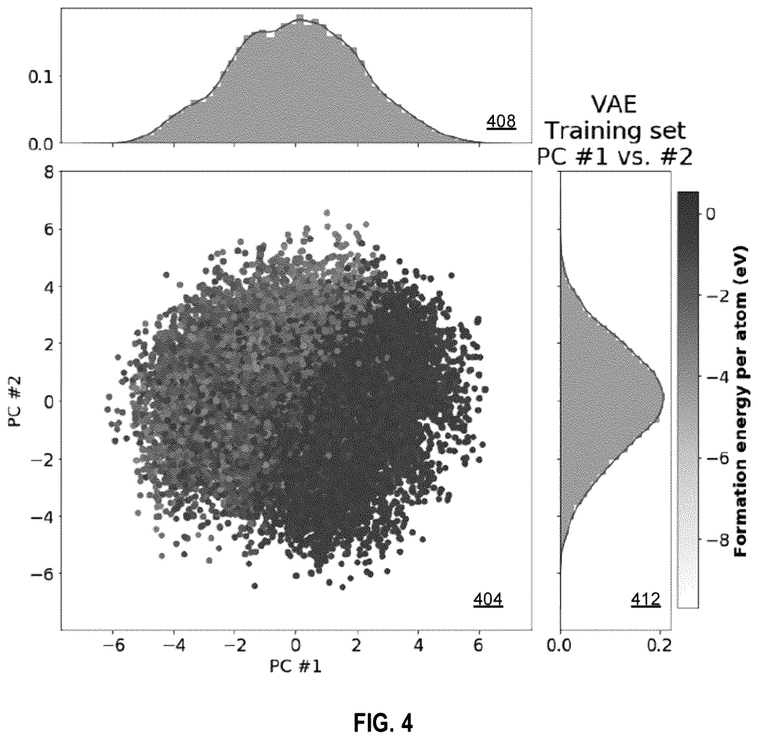

FIG. 4 shows that the latent space is ordered in the learned property of formation energy. Formation energy is a property that can be learned at stage 212. FIG. 4 shows a graph 404 with the x-axis showing principal component 1 and the y-axis showing principal component 2 of the latent embedding. The intensity of the data points in graph 404 is related to the formation energy per atom. A darker blue shows a higher formation energy, and a lighter blue shows a more negative formation energy. Graphs 408 and 412 show the distribution of datapoints along the principal components. FIG. 4 shows that the latent space can be continuous and ordered with respect to a learned property, such as formation energy. Thus, latent space 216 can be ordered in the VAE input space (structural and compositional mass density representation of the materials) and also in the property space.

Within latent space 216, regions or positions associated with high synthesizability and/or other properties can be identified. In some embodiments, these regions or positions may be valleys in the latent space. In some embodiments, the regions or positions may be identified as being in proximity to materials known to have high synthesizability and/or desired other properties. In some embodiments, the regions or positions may be identified by interpolating between materials known to have high synthesizability and/or desired other properties. Materials 220 within these regions or at these positions may be materials that are predicted to be synthesizable. However, the specific materials cannot be directly identified from the region in latent space 216. Transformations are performed to determine the crystal structures of materials 220.

At stage 224, a blurred voxel representation of a material in a region with high synthesizability and/or other properties is generated. The blurred voxel representation indicates the mass density at different coordinates. The blurred voxel representation may be decoded from latent space 216 using a variational autoencoder (e.g., DFC-prop-VAE). The same variational autoencoder used to encode the voxel representation may be used to decode. Images for the voxel representation and decoded blurred voxel representation are taken from Court (2020).

The variational autoencoder may be configured with the Encoder network that transforms representations of crystals (e.g., voxel representations) into the embedding space distribution (e.g., mean and standard deviation of embedded representations) and a reconstruction Decoder network that is configured to transform representations sampled from the embedding space distribution back into the initial-space representations of crystals.

Then the trained Decoder network from the variational autoencoder can be used to transform points of interest (e.g., associated with predictions satisfying one or more conditions) to the voxel representations, such that the crystal structures can be identified. The points of interest may correspond to (for example) a local or absolute maximum of a given predicted value, a local or absolute minimum of a given predicted value, a given local or absolute maximum or a score that depends on multiple predicted values, etc.

At stage 228, a model is used to predict the crystal structure from the blurred voxel representation. The model may take as an input electron density maps (i.e., voxel representation) and convert them to atomic sites in Cartesian coordinates or other coordinate systems. As an example, the blurred voxel representation may be input into 3D image segmentation networks, such as a 3D-UNET, SegNET, RefineNet, and DeepLab architecture. The 3D-UNET architecture may encode the blurred voxel representation. The 3D-UNET architecture may then decode to output two matrices: a binary mask of atomic presence for the given voxel and the species' identity matrix for the given voxel. The matrices are then used to generate the decoded crystal structure at stage 232. This can be performed using techniques described by Court et al., J. of Chemical Information and Modeling (2020), the entire content of which is incorporated herein by reference for all purposes. Other image segmentation models may also be used.

The 3D-UNET architecture is a convolutional neural network for 3D image segmentation. The architecture includes an analysis path and a synthesis path. In the analysis path, each layer may include two 3×3×3 convolutions followed by a ReLU. A 2×2×2 max pooling may follow. In the synthesis path, each layer may include an up-convolution of 2×2×2 and then two 3×3×3 convolutions followed by a ReLU. A 1×1×1 convolution reduces the number of output channels to the number of labels (i.e., 3). The 3D-UNET architecture is described in Cicek, O. et al., arXiv (2016), the entire contents of which are incorporated herein for all purposes.

### B. Crystal Surface Generative Model

Second generative model 108 may generate representations of crystal surfaces and/or adsorption sites. Representations of the crystal surfaces may include identities of the atoms in the crystal surface, bond lengths, or bond angles. The latent space can be navigated to identify candidate crystal surfaces and/or adsorption sites. Second generative model 108 may generate surfaces and/or adsorption sites with desired adsorption energies directly. In some embodiments, adsorption energy prediction framework 112 separate from second generative model 108 may not be needed.

However, second generative model 108 does not need to include a variational autoencoder. Second generative model 108 may use as an input an output of crystal structure from first generative model 104. In some embodiments, crystal surface representations may be generated by “slicing” a crystal structure along a plane. A candidate crystal structure may be analyzed, which may include predicting and/or analyzing the bond energies, bond lengths, and atoms of the crystal structure. Probabilities of different surfaces existing based on the analysis may be predicted. Surfaces with the highest probabilities or probabilities over a certain threshold (e.g., 50%, 60%, 70%, 80%, 90%, 95%, 99%) may be selected and determining what surfaces are likely to be present based on the characteristics (e.g., bond energies, bond lengths, atoms) of the crystal structure. The crystal surface may be constrained to be along a plane that goes through bonds having less than a certain bond energy.

Training a model may use data representing crystal surfaces and absorption sites on those surfaces.

### C. Adsorption Energy Prediction Framework

In adsorption energy prediction framework 112, adsorption energy may be predicted for a given catalyst and polymer using a model that relates adsorption energies of catalyst surfaces with polymers. Models that predict adsorption energies for catalysts and polymers may be developed using databases (e.g., The Open Catalyst Project) of adsorption energies of small molecules on catalyst surfaces. For example, models may predict adsorption energies for monomers, dimers, trimers, or oligomers, in place of adsorption energies of small adsorbates in databases. New models and databases may be created using DFT.

Adsorption energy prediction framework 112 may be integrated with second generative model 108. As an example, adsorption energy prediction framework 112 may involve a convolutional variational autoencoder, as in Ren et al., Matter (2022). The fingerprint may include multiple parts: crystal descriptor, surface descriptor, and/or site descriptor. For the crystal descriptor, the real-valued fingerprint part may be obtained from existing publications (e.g., Ren (2022)) or databases. For the surface descriptor, a one-hot encoded matrix of Miller indices (this means that only low-index facets are considered) may be constructed. Alternatively, hkl triplet integer indices may be included as descriptors instead of a one-hot encoded matrix. For the adsorption site, the coordinates and expansions of symmetry functions of local atomic environment (e.g., smooth overlap of atomic positions (SOAP), radial distribution function (RDF), angular distribution function (ADF)) of the site-neighboring atoms may be included. Surface and site fingerprints may be embedded into the representation instead of the reciprocal-space features as in Ren (2022). The target learning branch may be used to predict adsorption energy of the specific adsorbate and the system. The Open Catalyst Dataset may be a source for the catalyst surfaces and adsorption energies.

### D. Catalyst Selection

At user-defined catalyst selection 116, certain ranges of adsorption energies are targeted for depolymerization using a catalyst and the substrate (i.e., the polymer). The adsorption energy should not be too weak or too strong. If the interaction is too weak, the catalyst has little effect (the substrate is not activated); on the other hand, if the binding is too strong, the substrate has difficulty dissociating from the catalyst.

Embodiments described herein focus on using adsorption energies as criteria and abstraction to identify candidate catalysts. Criteria involving reaction mechanisms and rate-determining steps (e.g., Brønsted-Evans-Polanyi principles) may be too complicated and time-consuming to estimate for a wide variety of possible catalysts. Additionally, other possible criteria for suitable catalysts may not be as widely applicable to the space of possible catalysts as adsorption energy. For example, the d-band center of metals relative to the Fermi level governs hydrogen chemisorption strength on their surfaces, while the oxygen p-band center relative to the Fermi level governs transition metal oxide reactivity, but these criteria do not apply to many catalysts systems.

Catalyst candidates may be selected from crystals having certain adsorption energies estimated from the adsorption energy prediction model. Certain adsorption energy ranges may be desired for certain types of polymers. A user may be able to enter in a desired adsorption energy and select candidate catalysts having the predicted adsorption energy. In some embodiments, a user may enter in a specific polymer into a computing system, and the desired adsorption energy range will be provided by the computing system. The user may consider other criteria, including cost and availability of catalysts or components to synthesize the catalysts. A computing system may further filter available catalysts by these other criteria.

### E. Example Methods

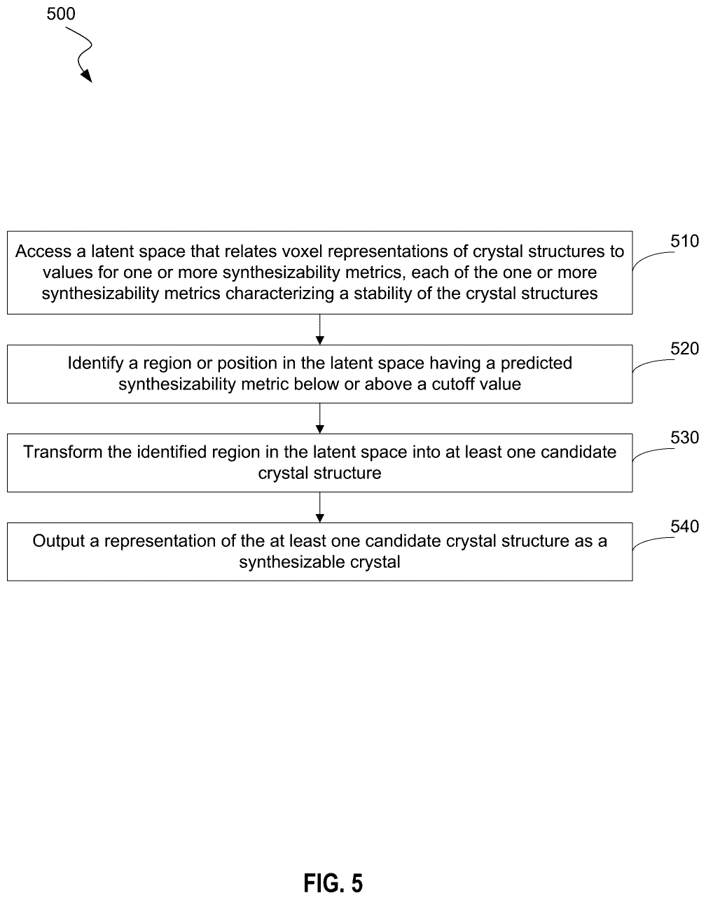

FIG. 5 is a flowchart of an example process 500 for predicting synthesizable crystals. In some implementations, one or more process blocks of FIG. 5 may be performed by a computing system, including computing system 2900 or any computing system described herein.

At block 510, process 500 may include accessing a latent space that relates voxel representations of crystal structures to values for one or more metrics. Each of the one or more metrics may characterize synthesizability or stability of the crystal structures. Both formation energy and energy above convex hull may relate to synthesizability and stability. The voxel representation may be similar to the voxel representation at stage 208 of FIG. 2. The latent space may be similar to latent space 216. The latent space may relate voxel representations of at least 100, 1,000, 10,000, 100,000, or 1,000,000 crystal structures to values for one or more metrics.

Each voxel representation of the voxel representations may include intensity values of voxels, and the intensity of each voxel may be related to a mass density at a spatial location of the voxel in the respective crystal structure. The voxel representation may correspond to spatial dimensions larger than a unit cell of a crystal structure. For example, the spatial dimensions may be a cube with a side length in the range of 3 to 5 Å, 5 to 7 Å, 7 to 8 Å, 8 to 10 Å, or greater than 10 Å. The voxel representation may include a voxel grid of x×y×z. In some embodiments, x=y=z, where x is an integer in a range from 10 to 20, 20 to 30, 30 to 40, 40 to 50, 50 to 60, 60 to 70, 70 to 100, or greater than 100.

The voxel representation may include one or more matrices. For example, the voxel representation may include a first matrix and a second matrix. The first matrix may specify the presence or absence of atoms at a given voxel, and the second matrix may specify the identity of the atomic species at the given voxel. Additional matrices may include local electron density of atoms, a matrix with unit cell length, and/or a matrix with coordinates of each voxel (e.g., as described in Court et al.).

The one or more metrics may characterize the synthesizability of a particular crystal structure. For example, the one or more metrics may include formation energy or hull energy. A low formation energy or hull energy may indicate that the reaction forming the crystal structure is thermodynamically favored, which suggests synthesizability. The one or more metrics may include a binary measure of synthesizability. The binary measure may indicate that a crystal structure is synthesizable when the crystal structure has experimental data (e.g., appears in a database, such as ICSD). A zero for the binary measure may indicate that the crystal structure is not synthesizable (or has not been attempted to be synthesized before).

The one or more metrics may characterize the stability of a particular crystal structure. For example, the one or more metrics may characterize a time the crystal structure is present at a given temperature and pressure before a certain amount of the crystal structure decomposes (e.g., a half-life). The one or more metrics may be based on kinetic rate constants for thermal decomposition or oxidation of the crystal structure. Some metrics may characterize both synthesizability and stability

In some embodiments, the one or metrics may include a singular metric that characterizes the overall synthesizability and/or stability of the crystal structure. For example, the singular metric may be a predicted probability that the crystal structure is synthesizable or stable. As an example, the singular metric may include the score described with FIG. 5.

The latent space may be generated by receiving crystal structure data of a plurality of crystals and receiving synthesizability data of the plurality of crystals. The plurality of crystals may include at least 100, 1,000, 10,000, 100,000, or 1,000,000 crystals. Crystal structure data may include unit cell values, element names, atom coordinates, chemical formula, chemical and physical properties, interatomic angles, bond distances, torsion angles, and/or details of diffraction experiments (e.g., temperature, pressure, experimental wavelength, radiation type, reflection data, intensity data). Crystal structure data may include any data in a CIF.

The synthesizability data may include the values for the one or more metrics. The synthesizability data may include null values for some of the one or metrics for some of the plurality of crystals as not every crystal may have all metrics available. The latent space may be generated by generating the voxel representations of the crystal structures using the crystal structure data. Additionally, the latent space may be generated by determining an embedded representation of each voxel representation of the voxel representations of the crystal structures. The encoding, property learning, and decoding may be performed with a variational autoencoder, including DFC-prop-VAE, as described with FIG. 2 and FIG. 5.

At block 520, process 500 may include identifying a region or position in the latent space having a predicted metric below or above a cutoff value or multiple predicted metrics above respective cutoff values. In some embodiments, target values for metrics may be defined based on input from a user and/or default settings. For example, a user may be able to adjust thresholds for each of one or more metrics in an interface. Target values may include a certain probability that a crystal structure is synthesizable or stable (e.g., at least 50%, 60%, 70%, 80%, 90%, 95%, or 99%). Stability may refer to a crystal structure that can exist at reaction conditions (e.g., standard temperature and pressure or within 10% of standard temperature and pressure) for a minimum duration (e.g., 1 second, 1 minute, 5 minutes, 10 minutes, 30 minutes, 60 minutes, or 24 hours). The regions or positions may be along a local or global maxima or minima in the latent space.

Identifying the region in the latent space may include using a variety of optimization algorithms. Such optimization algorithms may include convex approaches such as gradient descent, conjugate gradient, or Broyden-Fletcher-Goldfarb-Shanno (BFGS). Optimization approaches may also include non-convex, black box function optimization approaches such as systematic grid-searches or Bayesian Optimization. Optimization approaches may include iterative methods such as Newton's method, ellipsoid method, quasi-Newton methods, or simultaneous perturbation stochastic approximation (SPSA).

Regions or positions in latent space may be identified using supervised learning algorithms. Supervised learning algorithms may include decision trees, ensembles (bagging, boosting, random forest), k-nearest neighbors, linear regression, naive Bayes, artificial neural networks, logistic regression, perceptron, relevance vector machine (RVM), or support vector machine (SVM).

At block 530, process 500 may include transforming the identified region in the latent space into at least one candidate crystal structure. Transforming the identified region in the latent space into the at least one candidate crystal structure may include transforming the identified region into a candidate voxel representation of the at least one candidate crystal structure. For example, blurred voxel representation at stage 224 of FIG. 2 may be a candidate voxel representation. The candidate voxel representation may be transformed into the at least one candidate crystal structure. Transforming the candidate voxel representation into the at least one candidate crystal structure may include using a 3D-UNET architecture. For example, the decoded crystal structure at stage 232 of FIG. 2 may be the candidate crystal structure. In some embodiments, 3D-UNET and DFC-prop-VAE may allow for simultaneous training and optimization.

At block 540, process 500 may include outputting a representation of the at least one candidate crystal structure as a synthesizable crystal. As an example, the output of first generative model 104 in FIG. 1 may be the representation of the at least one candidate crystal structure. In some embodiments, the at least one candidate crystal structure may not be a crystal structure used to generate the latent space. In some embodiments, the at least one candidate crystal structure may be used to generate the latent space but the at least one candidate structure may be missing values for some of the one or more metrics (e.g., a score for synthesizability or stability).

Process 500 may be used for identifying possible catalysts for depolymerization. For example, process 500 may further include identifying a polymer of interest for decomposition. The polymer may be any polymer described herein. Process 500 may also include predicting an adsorption energy value of the polymer on the at least one candidate crystal structure. Predicting the adsorption energy value may be similar to adsorption energy prediction framework 112 of FIG. 1.

Process 500 may include accessing a latent space that relates the representation of the crystal structure to adsorption energy values characterizing the depolymerization power of the crystal structure. A region or position in the latent space having predicted adsorption energy values within a target range may be identified. The identified region in the latent space may be transformed into at least one candidate crystal structure. The representation of the at least one candidate crystal structure may be outputted.

The adsorption energy value may be compared to a threshold energy value. The comparison may be similar to the comparison described with user-defined manual catalyst selection 116. Identifying the at least one candidate crystal structure may include determining the adsorption energy value exceeds the threshold energy value. The threshold energy value may be a minimum energy value so that a reactant polymer will have a strong enough bond to the crystal structure. The adsorption energy value of the at least one candidate crystal structure then should be greater than the minimum energy value. In some embodiments, the threshold energy value may be a maximum energy value so that the product can desorb from the crystal structure. In these cases, the adsorption energy value should be less than the maximum energy value. In some embodiments, the adsorption energy value may be compared to a first threshold energy value and a second threshold energy value to determine that the adsorption energy value is above the minimum energy value and below the maximum energy value. Process 500 may include outputting the at least one candidate crystal structure as a prospect for depolymerizing the polymer using the comparison.

Process 500 may include generating a crystal surface and adsorption sites (e.g., second generative model 108) for depolymerization. Process 500 may further include generating a representation of a candidate crystal surface of the at least one candidate crystal structure. The representation of the candidate crystal surface may be generated by slicing the candidate crystal structure along a plane. Process 500 may include accessing a latent space that relates the representation of the crystal surface and/or adsorption sites to adsorption energy values characterizing the depolymerization power of the surface and/or site. A region or position in the latent space having predicted adsorption energy values within a target range may be identified. The identified region in the latent space may be transformed into at least one candidate crystal surface and/or adsorption site. The representation of the crystal surface and/or adsorption site may be outputted. In some embodiments, the crystal structure corresponding to the crystal surface and/or adsorption site may be classified as synthesizable or not using blocks 510 and 520.

A candidate adsorption site of the candidate crystal surface may be predicted using the representation of the candidate crystal surface, sites, and a model. The model may be trained by receiving a first set of data including training representations of crystal surfaces. The model may also be trained by receiving a second set of data indicating a plurality of training adsorption sites for the polymer. The model may further be trained by optimizing parameters of the model based on outputs of the model matching or not matching the training adsorption site in the second set of data when the first set of data is input to the model. An output of the model may specify an adsorption site. Process 500 may further include identifying the candidate adsorption site on the crystal surface. Predicting the adsorption energy value of the polymer on the at least one candidate crystal structure may include predicting the adsorption energy value of the polymer at the candidate adsorption site.

Process 500 may include synthesizing the at least one candidate crystal structure. In some embodiments, the at least one candidate crystal structure may be limited to containing no more than three elements. In some embodiments, the at least one candidate crystal structure may be a zeolite, including any zeolite or group of zeolites in Arroyave et al., J. Am. Chem. Soc. (2022); Kts et al., ACS Catal. (2021); and Zichittella, JACS Au (2022). Process 500 may include depolymerizing the polymer using the at least one candidate crystal structure. The synthesized candidate crystal structure may be contacted with the polymer under target reaction conditions (e.g., temperature, pressure, reactants). In some embodiments, a polymer may be depolymerized in silico under the target reaction conditions using the at least one candidate crystal structure.

Process 500 may include Bayesian optimization in navigating the latent space. The latent space may be generated by constructing a predictive function to predict values for the one or more metrics from the embedded representations of the crystal structures, crystal surfaces, or adsorption sites. Constructing the predictive function may use the embedded representations and the synthesizability data. Generating the latent space may include evaluating a utility function that transforms a given point within the latent space into a utility metric that represents a degree to which identifying an experimentally derived synthesizability value for the given point is predicted to facilitate training a more accurate version of the predictive function. Based on the utility function, one or more particular points within the latent space may be identified as corresponding to utility metric values above a utility threshold value. The one or more particular points may be transformed into crystal structures. The identity of the crystal structures may be outputted. The crystal structures may then be experimentally analyzed to determine synthesizability and/or other properties.

Process 500 may include additional implementations, such as any single implementation or any combination of implementations described and/or in connection with one or more other processes described elsewhere herein.

Although FIG. 5 shows example blocks of process 500, in some implementations, process 500 may include additional blocks, fewer blocks, different blocks, or differently arranged blocks than those depicted in FIG. 5. Additionally, or alternatively, two or more of the blocks of process 500 may be performed in parallel.

### II. System Environment

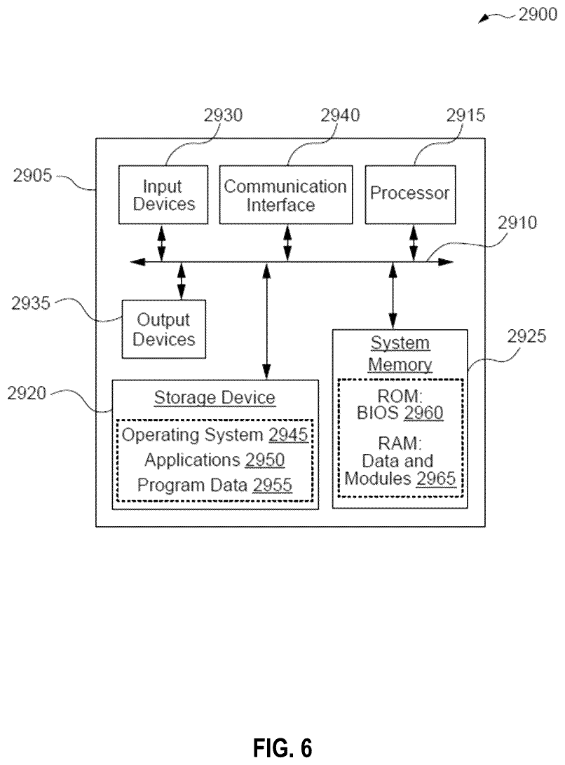

FIG. 6 is an illustrative architecture of a computing system 2900 implemented as some embodiments of the present disclosure. The computing system 2900 is only one example of a suitable computing system and is not intended to suggest any limitation as to the scope of use or functionality of the present disclosure. Also, computing system 2900 should not be interpreted as having any dependency or requirement relating to any one or combination of components illustrated in computing system 2900.

As shown in FIG. 6, computing system 2900 includes a computing device 2905. The computing device 2905 can be resident on a network infrastructure such as within a cloud environment, or may be a separate independent computing device (e.g., a computing device of a service provider). The computing device 2905 may include a bus 2910, processor 2915, a storage device 2920, a system memory (hardware device) 2925, one or more input devices 2930, one or more output devices 2935, and a communication interface 2940.

The bus 2910 permits communication among the components of computing device 2905. For example, bus 2910 may be any of several types of bus structures including a memory bus or memory controller, a peripheral bus, and a local bus using any of a variety of bus architectures to provide one or more wired or wireless communication links or paths for transferring data and/or power to, from, or between various other components of computing device 2905.

The processor 2915 may be one or more processors, microprocessors, or specialized dedicated processors that include processing circuitry operative to interpret and execute computer readable program instructions, such as program instructions for controlling the operation and performance of one or more of the various other components of computing device 2905 for implementing the functionality, steps, and/or performance of the present disclosure. In certain embodiments, processor 2915 interprets and executes the processes, steps, functions, and/or operations of the present disclosure, which may be operatively implemented by the computer readable program instructions. For example, processor 2915 can retrieve, e.g., import and/or otherwise obtain or generate candidate catalyst structures, encode crystal information into an embedding space, decode a point in an embedding space into a crystal, construct predictive functions, and evaluate utility functions. In embodiments, the information obtained or generated by the processor 2915 can be stored in the storage device 2920.

The storage device 2920 may include removable/non-removable, volatile/non-volatile computer readable media, such as, but not limited to, non-transitory machine readable storage medium such as magnetic and/or optical recording media and their corresponding drives. The drives and their associated computer readable media provide for storage of computer readable program instructions, data structures, program modules and other data for operation of computing device 2905 in accordance with the different aspects of the present disclosure. In embodiments, storage device 2920 may store operating system 2945, application programs 2950, and program data 2955 in accordance with aspects of the present disclosure.

The system memory 2925 may include one or more storage mediums, including for example, non-transitory machine readable storage medium such as flash memory, permanent memory such as read-only memory (“ROM”), semi-permanent memory such as random access memory (“RAM”), any other suitable type of non-transitory storage component, or any combination thereof. In some embodiments, an input/output system 2960 (BIOS) including the basic routines that help to transfer information between the various other components of computing device 2905, such as during start-up, may be stored in the ROM. Additionally, data and/or program modules 2965, such as at least a portion of operating system 2945, program modules, application programs 2950, and/or program data 2955, that are accessible to and/or presently being operated on by processor 2915, may be contained in the RAM. In embodiments, the program modules 2965 and/or application programs 2950 can comprise, for example, a processing tool to identify and annotate crystal structure data, a metadata tool to append data structures with metadata, and one or more encoder networks and/or encoder-decoder networks to predict properties, which provides the instructions for execution of processor 2915.

The one or more input devices 2930 may include one or more mechanisms that permit an operator to input information to computing device 2905, including, but not limited to, a touch pad, dial, click wheel, scroll wheel, touch screen, one or more buttons (e.g., a keyboard), mouse, game controller, track ball, microphone, camera, proximity sensor, light detector, motion sensors, biometric sensor, and combinations thereof. The one or more output devices 2935 may include one or more mechanisms that output information to an operator, such as, but not limited to, audio speakers, headphones, audio line-outs, visual displays, antennas, infrared ports, tactile feedback, printers, or combinations thereof.

The communication interface 2940 may include any transceiver-like mechanism (e.g., a network interface, a network adapter, a modem, or combinations thereof) that enables computing device 2905 to communicate with remote devices or systems, such as a mobile device or other computing devices such as, for example, a server in a networked environment, e.g., cloud environment. For example, computing device 2905 may be connected to remote devices or systems via one or more local area networks (LAN) and/or one or more wide area networks (WAN) using communication interface 2940.

As discussed herein, computing system 2900 may be configured to train an encoder-decoder network to predict properties from a voxel representation of a crystal structure. In particular, computing device 2905 may perform tasks (e.g., process, steps, methods and/or functionality) in response to processor 2915 executing program instructions contained in non-transitory machine readable storage medium, such as system memory 2925. The program instructions may be read into system memory 2925 from another computer readable medium (e.g., non-transitory machine readable storage medium), such as data storage device 2920, or from another device via the communication interface 2940 or server within or outside of a cloud environment. In embodiments, an operator may interact with computing device 2905 via the one or more input devices 2930 and/or the one or more output devices 2935 to facilitate performance of the tasks and/or realize the end results of such tasks in accordance with aspects of the present disclosure. In additional or alternative embodiments, hardwired circuitry may be used in place of or in combination with the program instructions to implement the tasks, e.g., steps, methods and/or functionality, consistent with the different aspects of the present disclosure. Thus, the steps, methods and/or functionality disclosed herein can be implemented in any combination of hardware circuitry and software.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

All patents, patent applications, publications, and descriptions mentioned herein are hereby incorporated by reference in their entirety for all purposes as if each individual publication or patent were specifically and individually indicated to be incorporated by reference and are incorporated herein by reference to disclose and describe the methods and/or materials in connection with which the publications are cited. None is admitted to be prior art.

What is claimed is:

1. A method comprising:
accessing a latent space that relates voxel representations of crystal structures to values for one or more metrics, each of the one or more metrics characterizing synthesizability or stability of the crystal structures;
identifying a region or position in the latent space having a predicted metric below or above a cutoff value;
transforming the identified region in the latent space into at least one candidate crystal structure; and
outputting a representation of the at least one candidate crystal structure as a synthesizable crystal.

2. The method of claim 1, further comprising:
identifying a polymer of interest for decomposition,
predicting an adsorption energy value of the polymer on the at least one candidate crystal structure using the representation of the at least one candidate crystal structure,
comparing the adsorption energy value to a threshold energy value, and
outputting the at least one candidate crystal structure as a prospect for depolymerizing the polymer using the comparison.

3. The method of claim 2, further comprising depolymerizing the polymer using the at least one candidate crystal structure.

4. The method of claim 2, wherein identifying the at least one candidate crystal structure comprises determining the adsorption energy value exceeds the threshold energy value.

5. The method of claim 2, further comprising:
generating a representation of a candidate crystal surface of the at least one candidate crystal structure,
predicting a candidate adsorption site of the candidate crystal surface using the representation of the candidate crystal surface and a model trained by:
receiving a first set of data including training representations of crystal surfaces,
receiving a second set of data indicating a plurality of training adsorption sites for the polymer, and
optimizing parameters of the model based on outputs of the model matching or not matching the training adsorption site in the second set of data when the first set of data is input to the model, wherein an output of the model specifies an adsorption site, and
identifying the candidate adsorption site on the crystal surface,
wherein:
predicting the adsorption energy value of the polymer on the at least one candidate crystal structure comprises predicting the adsorption energy value of the polymer at the candidate adsorption site.

6. The method of claim 1, wherein transforming the identified region in the latent space into the at least one candidate crystal structure comprises:
transforming the identified region into a candidate voxel representation of the at least one candidate crystal structure, and
transforming the candidate voxel representation into the at least one candidate crystal structure.

7. The method of claim 6, wherein transforming the candidate voxel representation into the at least one candidate crystal structure comprises using a 3D-UNET architecture.

2. The method of claim 1, wherein:
each voxel representation of the voxel representations comprises intensity values of voxels, and
the intensity value of each voxel is related to a mass density at a spatial location of the voxel in the respective crystal structure.

9. The method of claim 1, wherein the one or more metrics comprise formation energy or hull energy.

10. The method of claim 1, further comprising synthesizing the at least one candidate crystal structure.

11. The method of claim 1, wherein the latent space is generated by:
receiving crystal structure data of a plurality of crystals,
receiving synthesizability data of the plurality of crystals, wherein the synthesizability data comprises the values for the one or more metrics,
generating the voxel representations of the crystal structures using the crystal structure data, and
determining an embedded representation of each voxel representation of the voxel representations of the crystal structures.

12. The method of claim 11, wherein the latent space is further generated by:
constructing a predictive function to predict values for the one or more metrics from the embedded representations of the crystal structures, wherein constructing the predictive function uses the embedded representations and the synthesizability data,
evaluating a utility function that transforms a given point within the latent space into a utility metric that represents a degree to which identifying an experimentally derived synthesizability value for the given point is predicted to facilitate training a more accurate version of the predictive function, and
identifying, based on the utility function, one or more particular points within the latent space as corresponding to utility metric values above a utility threshold value.

3. A system comprising:
one or more data processors; and
a non-transitory computer readable storage medium containing instructions which, when executed on the one or more data processors, cause the one or more data processors to perform a set of actions including:
accessing a latent space that relates voxel representations of crystal structures to values for one or more metrics, each of the one or more metrics characterizing synthesizability or stability of the crystal structures;
identifying a region or position in the latent space having a predicted metric below or above a cutoff value;
transforming the identified region in the latent space into at least one candidate crystal structure; and
outputting a representation of the at least one candidate crystal structure as a synthesizable crystal.

14. The system of claim 13, wherein the set of actions further includes:
identifying a polymer of interest for decomposition,
predicting an adsorption energy value of the polymer on the at least one candidate crystal structure using the representation of the at least one candidate crystal structure,
comparing the adsorption energy value to a threshold energy value, and
outputting the at least one candidate crystal structure as a prospect for depolymerizing the polymer using the comparison.

15. The system of claim 14, wherein the set of actions further includes depolymerizing the polymer using the at least one candidate crystal structure.

16. The system of claim 14, wherein identifying the at least one candidate crystal structure comprises determining the adsorption energy value exceeds the threshold energy value.

17. The system of claim 14, wherein the set of actions further includes:
generating a representation of a candidate crystal surface of the at least one candidate crystal structure,
predicting a candidate adsorption site of the candidate crystal surface using the representation of the candidate crystal surface and a model trained by:
receiving a first set of data including training representations of crystal surfaces,
receiving a second set of data indicating a plurality of training adsorption sites for the polymer, and
optimizing parameters of the model based on outputs of the model matching or not matching the training adsorption site in the second set of data when the first set of data is input to the model, wherein an output of the model specifies an adsorption site, and
identifying the candidate adsorption site on the crystal surface,
wherein:
predicting the adsorption energy value of the polymer on the at least one candidate crystal structure comprises predicting the adsorption energy value of the polymer at the candidate adsorption site.

18. The system of claim 13, wherein transforming the identified region in the latent space into the at least one candidate crystal structure comprises:
transforming the identified region into a candidate voxel representation of the at least one candidate crystal structure, and
transforming the candidate voxel representation into the at least one candidate crystal structure.

19. The system of claim 13, wherein transforming the candidate voxel representation into the at least one candidate crystal structure comprises using a 3D-UNET architecture.

20. A computer-program product tangibly embodied in a non-transitory machine-readable storage medium, including instructions configured to cause one or more data processors to perform a set of actions including:
accessing a latent space that relates voxel representations of crystal structures to values for one or more metrics, each of the one or more metrics characterizing synthesizability or stability of the crystal structures;
identifying a region or position in the latent space having a predicted metric below or above a cutoff value;
transforming the identified region in the latent space into at least one candidate crystal structure; and
outputting a representation of the at least one candidate crystal structure as a synthesizable crystal.
